(19) United States
(12) Patent Application Publication
(10) Pub. No.: US 2009/0053288 A1
(43) Pub. Date: Feb. 26, 2009

(54) Hemostatic woven fabric

(76) Inventors: E. Stan Eskridge, JR., Chapel Hill, NC (US); Thomas H. Fischer, Hillsborough, NC (US); James V. Snipes, Clemmons, NC (US)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 12/228,490

(22) Filed: Aug. 13, 2008

Related U.S. Application Data
(60) Provisional application No. 60/965,431, filed on Aug. 20, 2007.

(51) Int. Cl. A61K 9/70; D03D 13/00; A61K 38/48; A61P 7/04; A61F 13/15

(52) U.S. Cl. 424/447; 442/203; 428/219; 424/94.64

(57) ABSTRACT

The present invention is directed to a woven fabric having the modified crowsfoot weave pattern shown in FIGS. 2A and 2B. The present invention is directed to a woven fabric comprising about 65 wt % fiberglass yarn and about 35 wt % bamboo yarn, the woven fabric (1) being about 15.0 ounces per square yard (OSY); (2) having a thread count of about 760; and (3) having the modified crowsfoot weave pattern shown in FIGS. 2A and 2B. Additional ingredients may also be added to the woven fabrics of the invention to enhance the hemostatic properties.

### CROSS-REFERENCE TO RELATED APPLICATIONS

This application claims the benefit of U.S. Provisional Application Ser. No. 60/965,431 filed Aug. 20, 2007.

### BACKGROUND OF THE INVENTION

1. Field of the Invention

The present invention is directed to weaves of fabrics, and more particularly to weaves of fabrics that demonstrate increased hemostatic response.

2. Brief Description of the Related Art

Despite considerable progress in understanding the pathophysiological processes involved in surface (topical) hemostasis, there remains an unmet need for materials that can be applied to staunch bleeding at sites of hemorrhage. Traumatic injury is the leading cause of death in the United States for individuals under 44 years of age, claiming 100,000 lives each year. In approximately half of these cases, exsanguination is the cause of death, and roughly 50,000 additional patients survive hemorrhaging injuries after massive red blood cell transfusion. The situation is equally critical in combat medical care. In a recent review of military casualties, the control of non-compressible bleeding was identified as the most important unmet need in military emergency medicine. Frequently the standard of care is use of a tourniquet to control “compressible” bleeding and the application of gauze to control the residual “noncompressible” bleeding. However, the haemostatic inefficiency of gauze is a major contributor to morbidity and mortality.

Textiles of various types have been used as wound dressings for many years, and the prior art is replete with examples of various bandages, gauzes, and wound dressings. For example, U.S. Pat. No. 4,173,131 discloses a lightweight, porous knitted elastic bandage produced from a warp of false-twist synthetic yarns with a filling inlay of regular yarns, and U.S. Pat. No. 6,015,618 discloses a composite yarn comprised of a chain stitch yarn knitted from a yarn A and at least an inlay yarn of a yarn B inserted into the chain stitch yarn along a longitudinal direction thereof. U.S. Pat. No. 3,419,006 (Dec. 31, 1968) discloses a wound dressing that contains a hydrophilic polymeric gel and an insoluble ether polymer. U.S. Pat. No. 4,323,061 discloses a two-part material for use in surgical bandages that is made from a combination of glass fibers and non-glass fibers.

Various compounds have also been added to bandages to aid a variety of healing processes. Two known examples of compounds that are included in bandages are antibiotics and compounds that increase the rate of hemostasis. For example, U.S. Pat. No. 3,328,259 (Jun. 27, 1967) discloses a dressing for a wound containing a hemostatic agent; U.S. Pat. No. 6,762,336 discloses hemostatic multilayer bandage that comprises a thrombin layer between two fibrinogen layers; and U.S. Pat. No. 4,616,644 discloses a hemostatic bandage having a thin coating of a high molecular weight polyethylene oxide.

While many additives have been used in conjunction with bandages to aid in wound healing, the particular bandage materials are usually conventional cotton gauze or other known material manufactured using conventional processes. In general, bandage materials are manufactured to provide high absorbance characteristics, but not necessarily high wound healing capabilities. There is a need in the art for a bandage with a weave that possesses hemostatic properties when applied to a wound. The present invention is believed to be an answer to that need.

### SUMMARY OF THE INVENTION

In one aspect, the present invention is directed to a woven fabric having a modified crowsfoot weave pattern.

In another aspect, the present invention is directed to a woven fabric comprising about 65 wt % fiberglass yarn and about 35 wt % bamboo yarn, the woven fabric (1) being about 15.0 ounces per square yard (OSY); (2) having a thread count of about 760; and (3) having a modified crowsfoot weave pattern.

In yet another aspect, the present invention is further directed to a woven fabric made by weaving about 65 wt % fiberglass yarn and about 35 wt % bamboo in a modified crowsfoot weave patter, the woven fabric being about 15.0 ounces per square yard (OSY) and having a thread count of about 760.

These and other aspects will become apparent upon reading the following detailed description of the invention.

### BRIEF DESCRIPTION OF THE FIGURES

The following description of the invention will be better understood when taken in conjunction with the following figures in which:

FIG. 1 shows a plain weave pattern of the prior art;

FIG. 2A shows one embodiment of the weave pattern of the fabric of the invention;

FIG. 2B shows another embodiment of the weave pattern of the fabric of the invention;

FIG. 3 shows shows a graph of blood volume loss over time using various weaves patterns including the weave pattern of the fabric of the invention;

FIG. 4 shows electrophoresis lanes depicting protein adsorption to materials;

FIG. 5 shows line graphs depicting thromboelastographic analysis of selected materials;

FIG. 6 shows bar graphs depicting thromboelastographic analysis of selected materials;

FIG. 7 shows scanning electron micrographs (SEMs) of selected materials;

FIG. 8 shows additional scanning electron micrographs (SEMs) of selected materials;

FIG. 9 shows a bar graph depicting quantification of RBCs with selected materials; and

FIG. 10 shows graphs depicting blood loss from vascular transection injuries.

### DETAILED DESCRIPTION OF THE INVENTION

The inventors of the present invention have unexpectedly found that a woven fabric having a modified crowsfoot weave pattern displays excellent hemostatic properties and fluid absorbency. The preferred materials used to make the woven fabric of the invention are textile denier fiberglass textured yarn and bamboo yarn. To further enhance the hemostatic properties of the hemostatic woven fabric, additional blood factors such as thrombin, lyophilized blood cells, lyophilized platelets, fibrin, fibrinogen, or combinations of these, may be added. These additional factors aid in activating the body's natural hemostasis cascade and result in a material that can rapidly arrest bleeding. The inventors have discovered that the hemostatic woven fabric of the invention rapidly arrests bleeding, and is useful in situations where large hemorrhages exist or when a patient cannot be immediately admitted to a hospital or trauma treatment center.

The hemostatic woven fabric of the present invention provides important advantages over current products that activate hemostasis. The present invention is capable of rapidly activating the body's natural hemostatic systems, such as the blood coagulation cascade, by providing what is believed to be a high locally surface area of materials that activate that cascade. In addition, by using lyophilized blood proteins, the hemostatic textile of the present invention may be stored in a dry state ready for immediate use for long periods of time. This aspect is particularly advantageous because previous products and systems required hydrated proteins for activation.

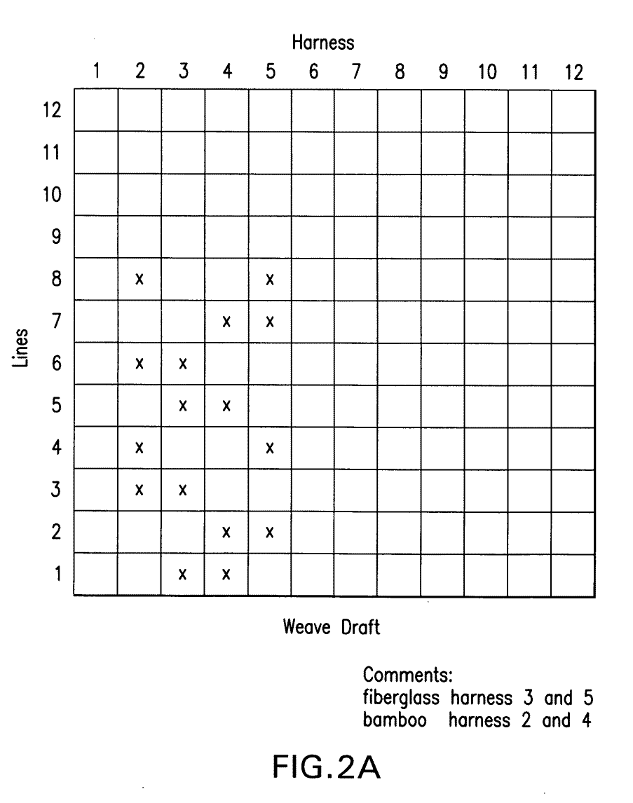

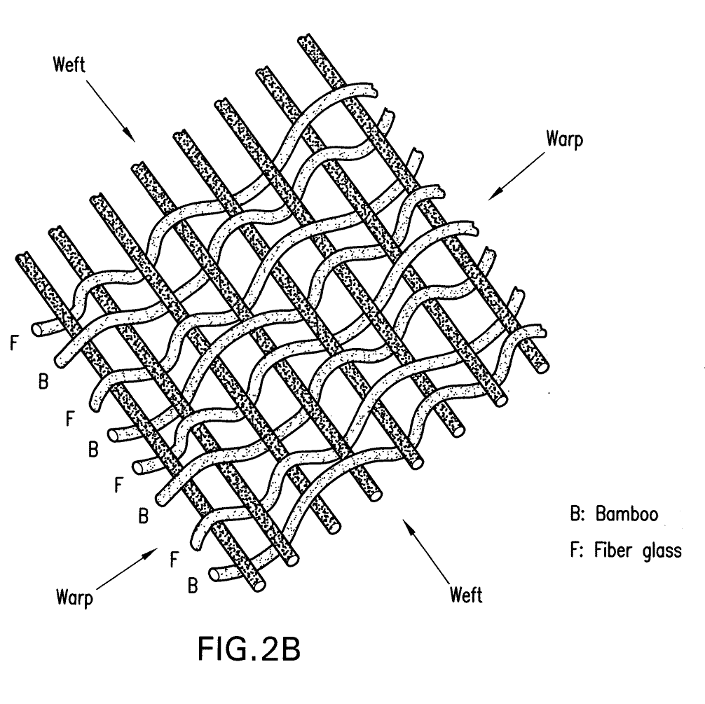

As defined herein, the phrase “modified crowsfoot weave pattern” refers to a weave that repeats on 8 picks wherein the warp yarn floats over two picks, goes under two picks, goes over one pick and under one pick, over one pick and under one pick then repeats, and wherein each end weaves the same but the harnesses are timed so that no progressive twill pattern is present, and has the patterns shown in FIGS. 2A and 2B.

As indicated above, one embodiment of the present invention is a woven fabric comprising about fiberglass yarn and bamboo yarn and having the weave patterns shown in FIGS. 2A and 2B.

The fiberglass yarn component is preferably a fiberglass prepared by extrusion or electrospinning processes, and has fiber diameters from 5 nanometers to 15 microns. Types of glass contemplated for use in the present invention include but are not limited to alumino-borosilicate glasses with low sodium oxide content, borosilicate glass, lead glass, aluminosilicate, alkali-barium silicate, vitreous silica, chalcogenide glass, phosphate glass, and bioactive glass sold under the trade name “BIOGLASS”. The dimensions of the glass fiber component may be described by conventional nomenclature, including the following designations: B (3.5 micron diameter); C (4.5 micron diameter); D (5 micron diameter); DE (6 micron diameter); E (7 micron diameter); G (9 micron diameter); H (10 micron diameter); or K (13 micron diameter). In addition, yarn yield of the glass fiber component can range from 90,000 yards per pound to 450 yards per pound. The grade of the glass fiber may be any of electrical grade (“E”), chemical grade (“C”), or high strength (“S”), and the filaments may be in any arrangement, for example continuous, staple, or textured. The fiberglass yarns may also be used single strand or in a plied state using 2 to 20 or more yarns. Fiberglass may be either in textile denier ranges or in roving sizes. Fiberglass material is available commercially from various suppliers such as PPG, and is available commercially as Grades G75, E-grade fiberglass, ECG 75 1/0 (electrical grade, continuous filament, G fiber diameter) and the like, using the designations described above.

The preferred fiberglass yarn is a textured fiberglass yarn, which offers advantages in the present invention. Textured fiberglass yarn is made from continuous filament yarns that are post-treated by passing the yarn bundle through an air jet that bulks and deforms the yarn bundle. This treatment is often used when manufacturing yarns for use in composites. The textured yarn effectively offers more fiber surface area without adding additional weight, and also adds thickness to the fabric to provide a soft fiberous surface, as opposed to a slick harder surface that is normal with fabrics woven with non-textured yarns. Textured fiberglass yarns thus offer advantages in terms of absorbancy and contact surface area that heretofore have not been appreciated in the current bandage art. One preferred textured fiberglass yarn is ETDE 11.6 TEXO OS (abbreviated herein as 11.6 Texo).

The bamboo yarn component is known, and may be made by conventional methods, including ring, open end (OE), rotor, or air jet spinning, and may have counts ranging from 1/1 to 100/1 Ne. For bamboo warp, a useful range of counts is from 2/1 to 18/1 or the equivalent yield in plies of yarns. For bamboo weft, a useful range of counts is from 4/1 to 22/1 or the equivalent yield in plies of yarns.

The relative amounts of fiberglass yarn and bamboo yarn can range, for example from about 10 to 90 wt % fiberglass yarn and about 10 to 90 wt % bamboo yarn, based on the total weight of the woven fabric. Preferable amounts of these materials range from about 45 to 85 wt % fiberglass yarn and about 15 to 55 wt % bamboo yarn, and more preferably from about 60 to 70 wt % fiberglass yarn to about 30 to 40 wt % bamboo yarn. An example of a useful proportions of fiberglass yarn and bamboo yarn in the woven fabric of the invention is about 65 wt % fiberglass yarn and about 35 wt % bamboo yarn.

Preferably, the materials are woven into a fabric having a weight range from 9 OSY (ounces per square yard) to 18 OSY. One preferred and useful weight is 15.0 OSY. The thread count of the fabric of the invention can range from about 250 to about 1000, with a preferred thread count being about 760. The woven fabric of the invention may be made using conventional manufacturing equipment and processes known in the art.

As known in the prior art, a regular twill weave including the crowsfoot has the warp yarns floating over two weft yarns then under two weft yarns and repeats. A 2-2 twill weave can be repeated on 4 picks (weft yarns). In contrast, the modified crowsfoot weave of the invention repeats on 8 picks. The warp yarn floats over two picks, goes under two picks, goes over one pick and under one pick, over one pick and under one pick, then repeats. Each end weaves the same but the harnesses are timed so that like a crows foot no progressive twill pattern is present. The pattern of the invention is exemplified in FIGS. 2A and 2B.

In one preferred embodiment, the woven fabric of the invention is a 15 ounce/square yard needle loom tape with a system 2 catch thread. The weave pattern is a 4 harness twill that is a modification of a classic crowfoot pattern. The combination of the weave pattern and the fact that 87% of the fabric mass is warp yarn results in maximizing fiberglass yarns on the fabric surface. In the warp, equal numbers of 11.6 Texo fiberglass and 4/1 bamboo yarns alternate. Warp density is 38 ends per inch. The mass of the warp is 74% fiberglass and 26% bamboo. In the weft, the fabric is woven with 10 double picks per inch of 8/1 bamboo. The weft is 100% bamboo and the weft comprises 12% of the fabric mass. The fabric overall has a mass of 65% fiberglass and 35% bamboo. The fiberglass yarns are bigger than the bamboo yarns and the bamboo yarns tend to be covered by the fiberglass. Using the denier system, the yarn sizes are 3849 denier fiberglass, 664 denier weft yarn bamboo, and 1328 denier warp yarn bamboo.

The woven fabric of the invention may also be treated with various agents that enhance its effectiveness. Detailed examples of additional agents are disclosed in copending U.S. Patent Application Publication 2007/0160653, and herein incorporated by reference in its entirety. Examples of additional agents include organic or inorganic compounds that are microstatic or microcidal; organic or inorganic compounds that covalently react with blood coagulation proteins; organic or inorganic compounds that covalently react with wounded tissue to form covalent bonds for enhanced adhesion to tissues; organic or inorganic compounds that polymerize to form a three-dimensional polymer network at or on the wound; imaging agents such as ultrasound contrast agents (e.g., gas-filled microbubbles, metallic nanoparticles, and the like), radio-opaque agents (e.g., iodinated small molecules such as iopromide, iodinated high molecular weight polymers, and the like), magnetic resonance probes (e.g., ferumoxide iron nanoparticles, superparamagnetic metallic nanoparticles, diethylenetriaminepentaacetate (DTPA)-chelated gadolinium, and polymers that contain DTPA-chelated gadolinium, and the like).

Further additional agents that may be included in the woven fabric of the invention include skin conditioners such as aloe vera, vitamin E, coenzyme Q, collagen, and the like; anti-inflammatory agents such as aspirin, ibuprofen, acetominophen, vitamin C, COX-2 inhibitors, steroids, and the like; analgesics such as lidocaine, tetrocaine, opiates, cocaine, antihistamines, and the like; antimicrobial or antifungal agents such as bacitracin, silver salts, iodide, and the like; vasoconstrictors such as epinepherine, norepinephrine, vasopressin, hemoglobin, endothelins, thromboxanes, NO scavengers, and the like; growth factors such as MMP inhibitors, PDGF, and the like; anti-scar agents such as IL-11, anti-keloid compounds, and the like; cauterizing agents that undergo an exothermic reaction upon rehydration such as zeolites; dehydrating agents that are hydroscopic such dextran; prothrombotic agents, such as zeolite, dextran sulfate, polyphosphate, mineral interfaces, phosphatidyl serine, calcium, and the like.

The woven fabric of the invention may also include additional factors that act to activate the body's natural hemostatic systems and thus aid in quickly arresting bleeding. Such additional factors include thrombin or a plasma fraction that includes thrombin, rehydrated lyophilized (RL) platelets, RL blood cells, fibrin, fibrinogen, and combinations of these. In one preferred embodiment, thrombin is incorporated into the textile to impart additional hemostatic action. The thrombin can be from any source (naturally isolated, recombinant, etc.) or may be in the form of a plasma fraction or serum that contains thrombin and additional coagulation factors such as factor XII, factor XIIa, factor XI, factor XIa, factor XIII, factor XIIIa, factor IX, factor IXa, factor VIII, factor VIIIa, factor vWF, factor V, factor Va, factor X, factor Xa, and combinations thereof, or other coagulation cofactors such as components of animal venom, such as reptilase, or vasoactive agents such as endothelins, thromboxanes, nitrous oxide (NO) scavengers, or combinations thereof. These factors, or any of the factors listed above, may be in a dry or liquid form when incorporated into the textile of the invention.

The thrombin contemplated for use with the woven fabric of the invention may take any form including highly purified thrombin Ia from human or animal sources, genetically modified plants, or other natural or recombinant protein expression systems. In addition, partially purified thrombin from human or animal sources, genetically modified plants, or other natural or recombinant protein expression systems may be used in the present invention. The thrombin contemplated for use in the present invention may also be contained in purified or partially purified serum or plasma. In one embodiment, the thrombin used in the fabric of the present invention is a partially purified serum fraction containing thrombin IIa.

The preferred amount of thrombin in the woven fabric of the invention ranges from about 0.01% by weight to about 10% by weight, based on the total weight of the dry fabric. More preferred amounts of thrombin included in the fabric of the invention range from about 0.05% by weight to about 7% by weight, and most preferably from about 0.1% by weight to about 5% by weight, all based on the total weight of the dry fabric.

In one embodiment, to produce a hemostatic fabric that includes thrombin, the fabric is soaked in a solution containing thrombin, and frozen and lyophilized. Preservatives such as glycerol, propanediol, polyoxyethylene glycol (PEG) trehalose, and the like, may be included in the soaking solution to prevent the textile from becoming brittle or chalky during lyophilization. In general, preservative concentrations in the thrombin solution range to a maximum of about 20% (v/v). In preferred embodiments, about 12% (v/v) glycerol is used.

In another preferred embodiment, one or more of rehydrated lyophilized (RL) platelets, RL blood cells, fibrin or fibrinogen are incorporated into the fabric to impart additional hemostatic action. Rehydrated lyophilized blood cells and rehydrated platelets and methods of their manufacture are known in the art. See, for example, U.S. Pat. Nos. 4,287,087; 5,651,966; 5,891,393; 5,902,608; 5,993,804; all incorporated by reference herein. Briefly, RL platelets are made by isolating the platelets, exposing them to a fixative such as formaldehyde, and drying. RL platelets may also be purchased commercially from Entegrion, Inc. (Research Triangle Park, N.C.) under the trade name “STASIX”. Methods of isolation and purification of fibrin and fibrinogen are also known in the art.

Briefly, to produce RL blood cells, blood can be obtained from healthy volunteers, following signed informed consent, in citrate-phosphate-dextrose with adenine (CPDA-1) and subjected to centrifugation at 1000×g for 20 min to obtain RBCs. The erythrocytes are diluted to a hematicrit=5% in phosphate buffered saline (PBS) and centrifuged at 2,000×g for 10 min. This step can be repeated two additional times to separate RBCs from plasma proteins. RBCs may then be cross-linked with glutaraldehyde (for glut-RL RBCs) or a mixture of paraformaldehyde and glutaraldehyde (for para-RL RBCs). Unreacted aldehyde can be removed from the RBCs by centrifugation (as for the removal of the cells from plasma proteins), and finally the cells are frozen and lyophilized at −30° C.

Fibrin and fibrinogen are also available commercially from various sources. For example, clinical grade material is sold under the tradename HAEMOCOMPLETTAN P from CSL Behring (Marburg, Germany) and TISSEEL from Baxter (Deerfield, Ill. USA). Research grade material is available from Enzyme Research Laboratories (South Bend, Ind. USA). Fibrin and fibrinogen may also be isolated according to procedures known in the art (e.g., van Ruijven-Vermeer I A, et al., Hoppe Seylers Z Physiol Chem. 360:633-7 (1979)). Fibrin and fibrinogen may also be isolated using glycine, ammonium sulfate, or ethanol precipitations that are known in the art.

The RL platelets, RL blood cells, fibrin, or fibrinogen, may be added in powder form by sprinkling or blowing the dried material onto the fabric and freeze-drying. Alternatively, these materials may be added to the fabric in solution form, and frozen and dried as described above. Preservatives such as glycerol, propanediol, polyoxyethylene glycol (PEG) trehalose, and the like, may be included in the soaking solution to prevent the textile from becoming brittle or chalky during lyophilization. In general, preservative concentrations in the thrombin solution range to a maximum of about 20% (v/v). In preferred embodiments, 12% (v/v) glycerol is used.

Any combination of RL blood cells, RL platelets, fibrin and/or fibrinogen may be incorporated into the fabric of the present invention. Preferably, the total amount of RL blood cells, RL platelets, fibrin and/or fibrinogen ranges from about 0.1% to about 50% based on the total weight of the dried fabric. In exemplary embodiments, the hemostatic textile of the invention may include the following combinations (all weight percents are expressed based on the total weight of the dried fabric):

| | RL Platelets or RL Blood | Fibrin or Fibrinogen |
|---|---|---|
| Range | Cells (wt %) | (wt %) |
| Preferred Range | 0.1 to 20 | 0.1 to 5 |
| More Preferred Range | 1.0 to 10 | 0.5 to 2 |
| Most Preferred Range | 3 to 7 | 0.75 to 1.5 |

In yet another embodiment, the fabric of the invention includes both thrombin or a fraction containing thrombin and one or more of rehydrated lyophilized (RL) platelets, RL blood cells, fibrin or fibrinogen. For example, one preferred combination of dried platelets, fibrinogen and thrombin is about 3 to 7 wt % RL platelets, 0.75 to 1.5 wt % fibrinogen, and 0.1 to 5 wt % thrombin, all based on the total weight of the dried fabric. In one particularly preferred embodiment, a combination of about 5 wt % RL platelets, about 1 wt % fibrinogen, and about 0.1 wt % thrombin is used.

A hemostatic woven fabric that contains both thrombin and one or more of rehydrated lyophilized (RL) platelets, RL blood cells, fibrin or fibrinogen is preferably made by first incorporating thrombin into the fabric followed by incorporation of one or more of rehydrated lyophilized (RL) platelets, RL blood cells, fibrin or fibrinogen using the techniques described generally above. In one embodiment, the hemostatic woven fabric of the invention may be infused with a combination of fibrinogen and thrombin as disclosed in U.S. Pat. No. 6,113,948, incorporated herein by reference, and available from ProFibrix BV (Leiderdorp, The Netherlands) under the trade name “FIBROCAPS” (a combination of fibrinogen microspheres and thrombin microspheres). Preservatives such as glycerol, propanediol, polyoxyethylene glycol (PEG) trehalose, and the like, may be included in the soaking solution to prevent the textile from becoming brittle or chalky during lyophilization. In general, preservative concentrations in the thrombin solution range to a maximum of about 20% (v/v). In preferred embodiments, 12% (v/v) glycerol is used.

Generally, the hemostatic woven fabric of the invention is made by the following steps:

1. RL platelets or RL blood cells are prepared and lyophilized according to published procedures;

2. The fabric is manufactured from textile components as described below. During this step, the fabric may be treated chemically by addition of a defined amount of agents such as glycerol, propanediol, polyoxyethylene glycol (PEG) to preserve the textile and aid in adhesion of hemostatic proteins. Additionally, in this step, thrombin-containing serum or plasma is freeze-dried onto the textile matrix.

3. Hemostatic proteins such as RL platelets, RL blood cells, fibrin, or fibrinogen are applied directly to a selected surface of the fabric (e.g., a surface that will contact wounded tissue) at a pre-selected particle density (protein per square area of textile surface) or weight percentage based on the total weight of the textile. The hemostatic proteins may be applied in any order, and may be applied to the textile in solution form or in a dry form. In one embodiment, RL platelets may be aldehyde-stabilized, applied to the fabric in a liquid state, and then freeze dried onto the textile.

4. The infused hemostatic fabric is packaged and optionally subjected to sterilization (e.g., gamma or UV irradiation).

The hemostatic woven fabric of the invention is capable of activating hemostatic systems in the body when applied to a wound, including the blood coagulation systems and vasoconstriction systems. It has long been known that various materials activate platelets and other blood coagulation factors when they come into contact with a wound site. Platelets, as a primary cellular component of blood that provide hemostasis in response to vascular injury, become contact-activated when exposed to foreign materials such as metals glass, and plastics. See, for example, Barr, H. The stickiness of platelets. Lancet ii, 775 (1941)). In addition, it is well known that thrombin converts fibrinogen to fibrin in the blood clotting cascade. The combination of components in the hemostatic woven fabric of the present invention act together locally and synergistically to activate the blood coagulation cascade in a highly concentrated and localized form when applied to a wound.

The hemostatic woven fabric of the invention is useful as a wound dressing, for example, a bandage, gauze, and the like, or may be shaped into sutures for use in surgery. Additional uses include forming the hemostatic woven fabric for use in the manufacture of protective clothing or liners for clothing, or for use in tourniquets. Additionally, in another embodiment, the fabric of the present invention is in the form of a kit for use in surgery or emergency or trauma situations. The kit includes the hemostatic fabric of the invention in rolls, sheets, or other appropriate shape, and may be used with or without the additional blood factors.

### EXAMPLES

The following examples are intended to illustrate, but in no way limit the scope of the present invention. All parts and percentages are by weight and all temperatures are in degrees Celsius unless explicitly stated otherwise.

### Example 1

### Analysis of Weaves

Various weaves of fiberglass and bamboo yarns were produced and evaluated for hemostatic capability. The parameters of each weave is shown in Table 1.

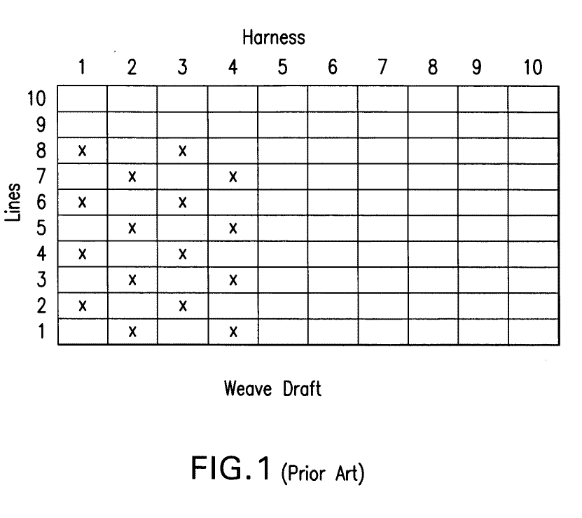

TABLE 1
Fiberglass and Bamboo Yarn Weaves

| | Weight | Bamboo | Fiberglass | | | | Warp Yarns | Weft Yarns | Thread | Weave |
|---|---|---|---|---|---|---|---|---|---|---|
| Sample | (OSY*) | (wt %) | (wt %) | Warp 1 | Warp 2 | Weft | per Inch | per Inch | Count | Pattern |
| 1 | 8.5 | 41.64 | 58.36 | 38 | 36 | 10 PI | 19 | 20 | 380 | Plain |
| | | | | 4/1 B | 11.6 Tex | 8/1 B | | | | (FIG. 1) |
| 2 | 8.34 | 20.27 | 79.73 | 36 | 38 | 10 | 19 | 20 | 380 | Plain |
| | | | | 4/1 B | 11.6 Tex | 75 1/0 Tex | | | | (FIG. 1) |
| 3 | 10.03 | 16.86 | 83.14 | 38 | 38 | 20 | 19 | 40 | 760 | Plain |
| | | | | 4/1 B | 11.0 Tex | 75 1/0 Tex | | | | (FIG. 1) |
| 4 | 10.34 | 52.06 | 47.94 | 38 | 38 | 20 | 19 | 40 | 760 | Plain |
| | | | | 4/1 B | 11.6 Tex | 8/1 B | | | | (FIG. 1) |
| 5 | 9.54 | 36.9 | 63.1 | 76 | None | 16.7 | 19 | 33.4 | 634.6 | Plain |
| | | | | 4/1 B | | 37 1/0 Tex | | | | (FIG. 1) |
| 6 | 10.72 | 32.85 | 67.19 | 76 | None | 20 | 19 | 40 | 760 | Plain |
| | | | | 4/1 B | | 37 1/0 Tex | | | | (FIG. 1) |
| 7 | 5.87 | 59.95 | 40.05 | 76 | None | 13 | 19 | 26 | 494 | Plain |
| | | | | 4/1 B | | 75 1/0 Tex | | | | (FIG. 1) |
| 8 | 10.45 | 33.6 | 66.3 | 76 | None | 13 | 19 | 26 | 494 | Plain |
| | | | | 4/1 B | | 25.0 Tex | | | | (FIG. 1) |
| 9 | 15.0 | 35.08 | 64.92 | 76 | 76 | 10 | 38 | 20 | 760 | Modified |
| | | | | 4/1 B | 11.6 Tex | 8/1 B | | | | Crowsfoot |
| | | | | | | | | | | (FIGS. 2A |
| | | | | | | | | | | and 2B) |

*Ounces per Square Yard

In Samples 1-8, a plain weave pattern as known in the prior art was used, where both warp and weft yarns alternate each pick and each end from up to down. The pattern of this plain weave is shown in FIG. 1 where X refers to a raised yarn and blank refers to a lowered yarn. Additionally, in the plain weave samples 1-8, all the warp ends alternate between faces with floats limited to one pick. Further, on the traditional crowsfoot weave, all warp ends float over two picks and in a random manner. The modified crowsfoot of Sample 9 combines two end floats with single floats on the same yarn and does so in a random manner. The modified crowsfoot of Sample 9 also provides that all filling floats are over two ends, which is in contrast to a traditional crowsfoot where alternate picks have single end floats. The weave pattern of Sample 9 is shown in FIGS. 2A and 2B.

Each of the above samples of woven fabric were evaluated for flow-stasis and hemostatic properties as follows.

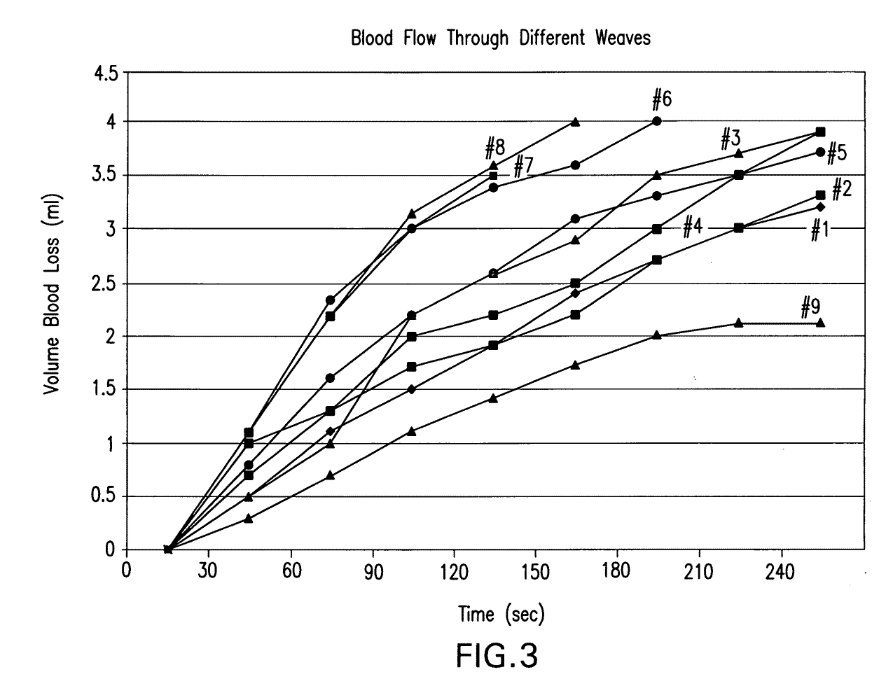

Whole venous blood was drawn from consenting healthy volunteers in accordance with institutional guidelines. Whole blood (42.5 ml blood to 7.5 ml anticoagulant, 3.2% w:v citrate, pH=7.4) was drawn into a syringe and used within two hours for the flow-stasis analysis. A 5 ml polypropylene syringe was rested in an upright position without application of pressure on pieces of dual fiber textile so that the bottom opening of the syringe was in contact with the upper surface of the textile. The analysis was initiated by adding 5.0 μl of 1.0 M calcium chloride stock to 5.0 ml blood, and then immediately placing blood in the syringe. The volume of blood that was lost through the bottom opening of the syringe through the materials was measured every thirty seconds by noting the blood level in the syringe. The results are shown in FIG. 3.

As shown in FIG. 3, weave Sample 9 displayed superior results in retarding blood flow. Complete flow cessation was obtain in approximately three minutes with weave Sample 9. In contrast, flow cessation did not occur within the four minute test period with the other weaves. The capability of weave Sample 9 to retard blood flow reflects the ability of the materials to accelerate platelet and humoral coagulation cascade activation faster than flow kinetics dilute the factors for the fibrin polymerization process.

Without wishing to be bound by any particular theory, it is believed that weave Sample 9 displays superior blood retardation and coagulation characteristics because the modified crowsfoot weave pattern exposes the maximum warp yarn surface in the final fabric. In addition, the use of textile denier fiberglass textured yarn allows more fiber surface directly in contact to the wound than could be possible with conventional untextured fiberglass yarns. Alternate yarns in the warp are 4/1 bamboo, and it is believed that the high surface exposure of fiberglass filaments in very close proximity to bamboo yarns contributes, at least in part, to the superior effectiveness of this weave.

### Example 2

### Analysis of Hemostasis

Weave Sample 9 produced and described above was further analyzed for hemostatic properties as follows:

### A. Materials and Methods

Whole blood and platelet rich plasma isolation—Peripheral blood from consented healthy volunteers was drawn into citrate anti-coagulant and immediately use for some experiments. Alternatively, platelet rich plasma was isolated with differential centrifugation as detailed elsewhere (Fischer T H, et al., Biomaterials 26:5433-5443 (2005)). The platelet concentration in the platelet rich plasma was measured with a Hiska haematological analyzer, and the platelet concentration was adjusted to 150,000 platelets/μl by diluting the sample with platelet free plasma.

Thrombin generation kinetics—The effect of fibers on the kinetics of thrombin generation in platelet rich plasma (at 150,000 platelets/μl) was investigated by following the hydrolysis of the thrombin substrate D-phe-pro-arg-ANSNH to yield a fluorescent reaction product as detailed elsewhere (Fischer T H, et al., J Biomed Mater Res A. 80:167-174 (2007)). A 300 μg sample of each fiber was tested in 100 μl platelet rich plasma in triplicate with the fluorogenic substrate D-phe-pro-arg-ANSNH. The time course for thrombin generation was initiated by adding CaCl₂ for 10 mM to each sample. The lag time for thrombin generation was defined as the time point at which the fluorescence increased 10% over the initial baseline value. Three independent measurements were performed with each fiber, and then statistical significance (p values) was inferred from standard deviations and mean values with student t-test with two-tail consideration.

Characterization of adsorbed plasma proteins—Glass, Sample 9, or gauze was incubated for 30 seconds at 50 mg/ml with normal single donor plasma that contained 10 mM CaCl₂. The materials were then removed and diluted in 50 ml of citrated saline. Bulk materials were diluted to 10 mg/ml in citated saline and centrifuged at 1000×g. Pellets were resuspended for the same material concentration and re-centrifuged. After a total of five centifugational washes the final material pellets were suspended at 100 mg/ml in reduced electrophoretic sample buffer and then incubated for five minutes at 100° C. to remove bound proteins from the materials. Electrophoresis was performed as known in the art on gels with 11% (w/v) polyacrylamide content. Selected bands were subjected to GC/mass spectrometry analysis for identification as described elsewhere (Fischer T, et al., In: Columbus F, ed. New Research on Biomaterials. Hauppauge, N.Y.: Nova Science Publishers, Inc.; 2006.).

Thromboelastography (TEG)—TEG measurements were performed with a TEG-5000 Thromboelastograph Hemostasis Analyzer (Haemoscope Corporation, Niles, Ill.). The assays were initiated by adding CaCl₂ for 10 mM to the whole blood, then the mixture (330 ul) was immediately transferred to the TEG chamber contained 1 mg of material in 20 μl citrated saline. The final fiber concentration was thus 3.0 mg/ml. Measurements were performed for two hour at 37° C., and then relevant parameters were extracted from the “stiffness” curve. Statistical significances (p values) were calculated with two tails from standard deviations and mean values with the student t test.

Scanning electron microscopy (SEM)—SEM analysis of the materials was performed as known in the art. Briefly, whole peripheral blood from volunteers was allowed to flow directly from the venipuncture butterfly onto each tested material so that each material was covered with excess blood. The materials were allowed to incubate for twenty seconds with the blood, and then the were added to 50 ml citrated saline with 1 mM EGTA to quench haemostatic processes. All samples were allowed to settle for five minutes with gravity, and then were rediluted with citrated saline. This process was repeated two more times to free each sample of unbound blood cells. After twenty seconds of contact with blood and multiple cycles of dilution and material/blood cell complex settling, glutaraldehyde was added for 0.1% (w/v) and the samples were allowed to incubate at room temperature for one hour. The samples were diluted 1/1 (v/v) with 4% paraformaldehyde for a final concentration of 2%, and then more glutaraldehyde was added for a final concentration of 0.5%. The initial stabilization step with 0.1% glutaraldehyde has been shown to minimize osmotically driven alterations in RBC morphology due to paraformaldehyde exposure (Fischer T H, et al., Microsc. Res. Tech. 65:62-71 (2004)). Samples were stored at 4° C. overnight and then examined with a Cambridge S200 scanning electron microscope at 20 kv.

Measurement of bound RBCs—10 mg samples of the dual fiber textile and gauze were directly exposed to 1.0 ml whole peripheral blood and washed as detailed for SEM. The samples were then placed in 10 ml of distilled water with 1% TX-100 to release hemoglobin from bound RBCs. The samples were centrifuged at 10,000×g for five minutes, then the absorbance at 414 nm was measured to quantify the total amount of hemoglobin (and thus number of RBCs) associated with each material. Three independent measurements were performed with each material.

Measurement of extent of RBC lysis due to material contact 10 mg samples of dual fiber textile and gauze were exposed to 1.0 ml whole peripheral blood as in the last two sections. After twenty seconds after exposure, the samples were centrifuged at 10,000×g for five minutes to pellet blood cells and other materials. The optical density at 414 nm was measured to quantify the amount of released hemoglobin in the supernatants. Three independent measurements were performed for each material.

Porcine brachial plexus and femoral artery transection hemorrhage models—40 to 50 kg mixed breed pigs were anesthetized with isofluorane and then several sensors were placed to follow hemodynamic and vasoactive processes: a pulmonary artery thermo-dilution catheter was inserted via the external jugular vein into a pulmonary artery; micromanometer-tipped catheters were positioned via the left femoral vessels into the right atrium and thoracic aorta.

The hemorrhage-challenge phase of the experiment was performed in two parts. First, the transectional laceration of bilateral brachial vasculature was performed. Both brachial arteries and two associated ˜3 mm diameter veins were surgically exposed and completely transected in a near simultaneous manner with a single scalpel stroke. The penetrating cut-down sites were completely packed with either gauze or the dual fiber material, and then pressure was held for six minutes. Packing was removed and the amount of shed blood was ascertained as described in the next paragraph. Both wounds were then repacked with the dual fiber material to stabilize the animals. The second part of the experiment proceeded by surgically exposing the dilateral femoral arteries, transecting them in a near simultaneous manner, packing them with dual fiber textile or gauze. Pressure was held for six minutes, and then materials were removed for shed blood determination.

The amount of blood that was shed into the original packing samples was measured by placing the dual fiber material or gauze into one liter of distilled water to lyse RBCs. After two hours of stirring at room temperature and storage overnight at 2° C., the optical density at 414 nm was measured to obtain the amount of released hemoglobin. This value was used to calculate the number of shed RBCs and volume of lost blood. Seven animals were studied in this manner for seven sets of bilateral brachial and seven sets of bilateral femoral comparisons. Statistical significances (p values) were calculated with two tails from standard deviations and mean values with the student t test.

### B. Results

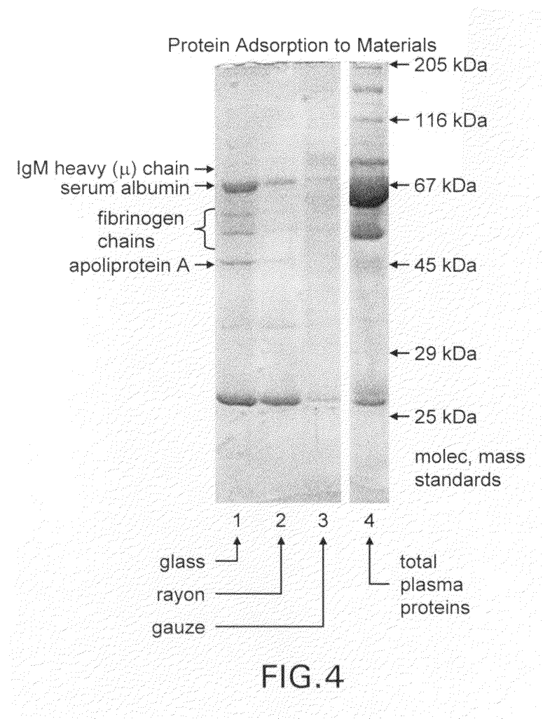

Plasma protein adsorption to materials The set of plasma proteins that tightly bind to each of glass, bamboo yarn (designated as “rayon” in FIG. 4), and gauze was characterized by incubating plasma with each material, exhaustively washing with citrated saline, and then performing protein electrophoresis. Briefly, glass fiber (lane 1), bamboo (“specialty rayon” (lane 2) or gauze (lane 3) was incubated with for 30 seconds with excess calcified normal human plasma. The materials were then bulk washed to remove unbound proteins, and then subjected to SDS-polyacrylamide gel electrophoresis to characterize bound proteins. Selected bands were identified with Western and/or GC-mass spectroscopic analysis as indicated. The results, which are representative of three independent determinations, show that each material bound a specific subset of plasma proteins. Based on the overall density of the protein stain, glass and the bamboo yarn bound more total proteins than gauze, including several proteins that were identified with GC/mass spectrometry analysis as the 70 kDa IgM heavy (μ) chain, 67 kDa serum albumin, and the 50-60 kDa fibrinogen chains.

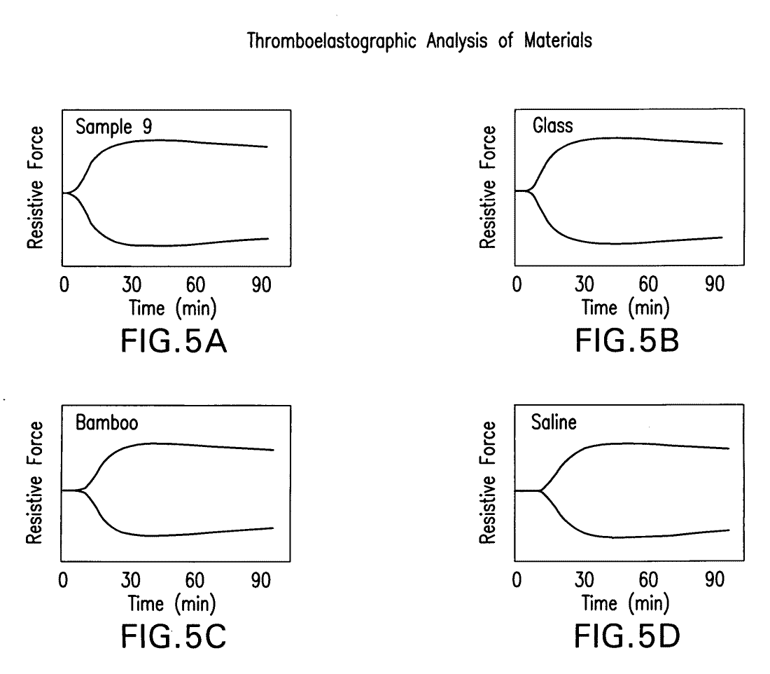

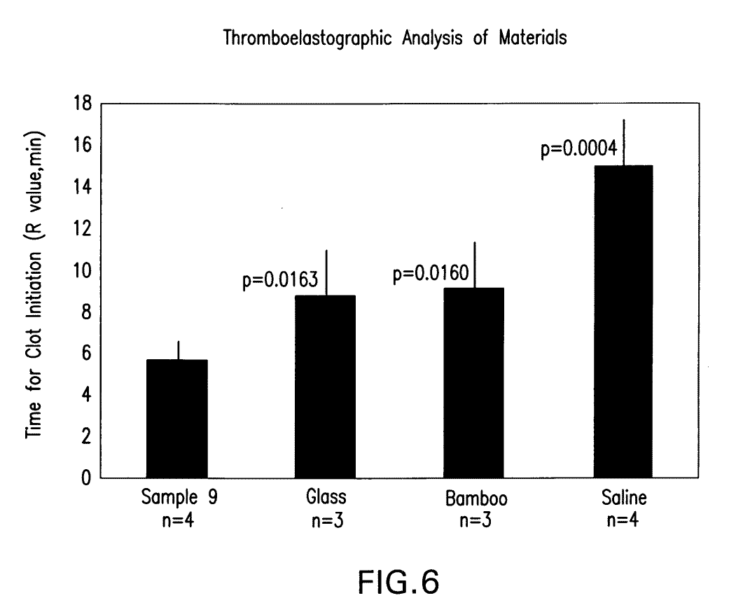

In vitro properties of the glass/specialty rayon dual fiber textile—Material of Sample 9 above, type E glass alone, and bamboo fiber alone were compared using thromboelastographic analysis with whole blood (see FIGS. 5 and 6). This analysis was performed with the same amount of total material (for 3 mg/ml glass, specialty rayon, or glass+specialty rayon) in each cuvettes. Sample 9 material (R=5.8+/−0.9 min, n=4 determinations) was found to accelerate fibrin clot formation in a statistically significant manner as compared to glass (R=8.8+/−1.3 min, n=3 determinations, p=0.00163 compared to Sample 9 material), bamboo fibers (R=9.1+/−1.5 min, n=3 determinations, p=0.0160 compared to Sample 9 material) or saline negative control (R=14.9+/−2.7 min, n=4 determinations, p=0.0004 compared to Sample 9 material). The coagulation activation observed with the saline samples reflected the interaction with the TEG cuvette surface material. Since the same amount of total material was in each cuvette, the acceleration of hemostasis measured with the dual fibers reflects a synergistic effect, not an effect that is additive for the two materials.

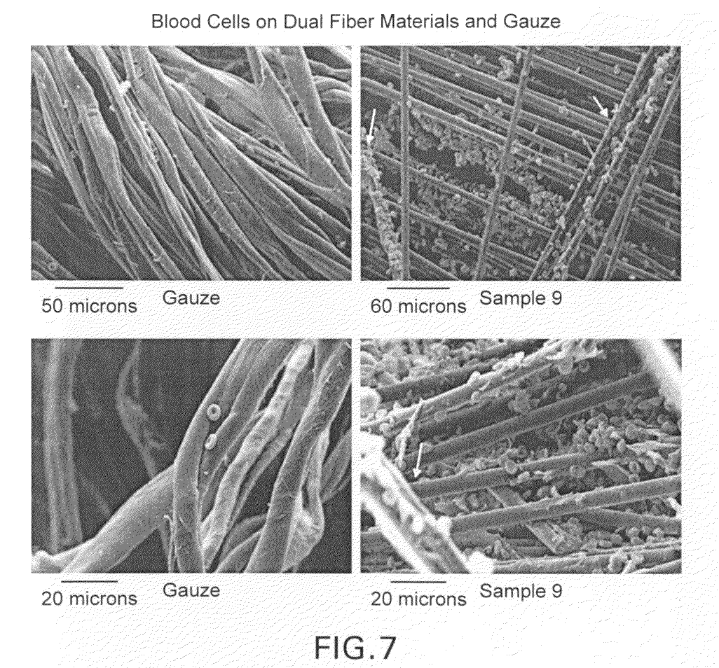

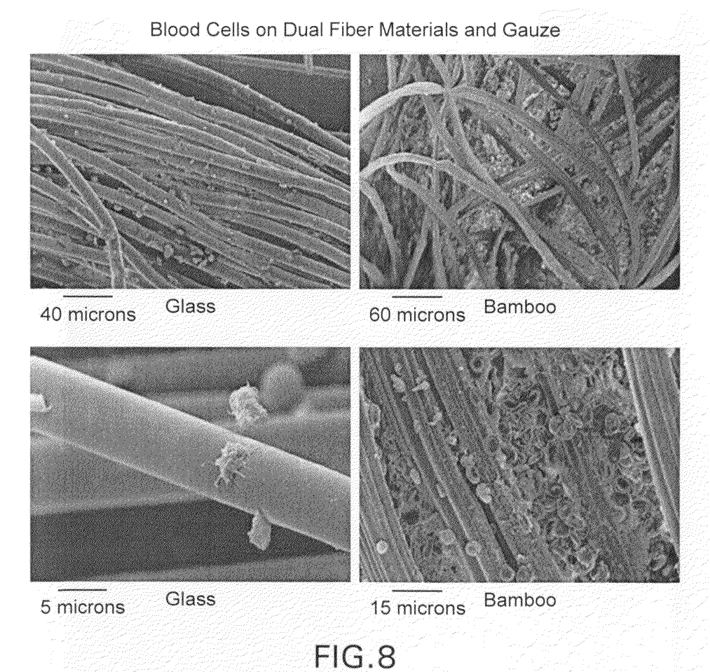

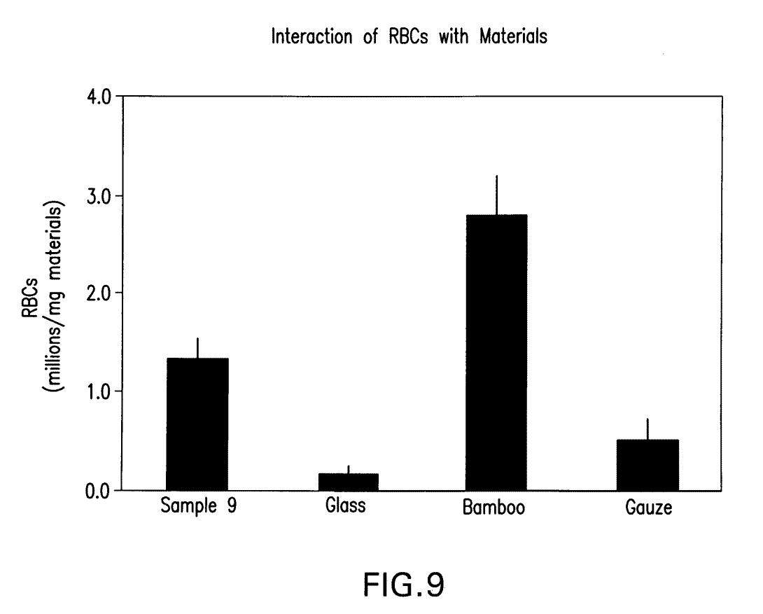

Analysis of the Sample 9 material and gauze by scanning electron microscopy (SEM) following contact with excess peripheral blood (see FIG. 7, which is representative of five independent SEM examinations) showed the glass/bamboo matrix tightly binding significant numbers of RBCs, while these cells only sparsely covered the gauze matrix. Glass filaments are recognizable as cylindrical filaments, while the specialty rayon has a more complex “ribbed” cross-sectional geometry SEM analysis of glass and specialty rayon separately indicated that glass robustly bound platelets in a highly activated morphology, while the specialty rayon bound and agglutinated RBCs (see FIG. 8). Quantification of the number of RBCs on each matrix (FIG. 9) revealed that the dual fiber textile of Sample 9 bound approximately three times as many RBCs per unit weight as gauze, and that the RBC binding to the dual fiber matrix of Sample 9 was due to the bamboo content. Significant lysis of RBCs did not occur (data not shown). These results indicate that the two materials in the dual fiber matrix of Sample 9 impart different but synergistic properties for accelerated clot formation.

Ability of the dual fiber bandage to provide hemostasis in porcine models—The ability of the glass/bamboo matrix of Sample 9 and gauze to limit blood loss from transected large vessel injuries in pigs was compared. The experiment was designed so that each animal served as its own control, and so that the animals presented a consistent hemodynamic and hemostatic profile characterized by a pre-hemorrhagic shock phase of injury. Two types of injuries were established on each of seven pigs. First, the brachial artery and two associated large veins in bilateral sides of the animals were completely transected in a near-simultaneous manner. This provided an exsanguinating hemorrhage that is both arterial and venous in nature. The bilateral cut-down/injury sites were immediately packed with as much dual fiber textile or gauze as required to completely fill each injury site. Pressure was then held for six minutes, and then blood loss onto packing materials and any shed blood was measured from each wound site. The bilateral brachial vasculature injury sites were then re-packed with dual fiber textile so as to stabilize the animal for the second set of femoral injuries.

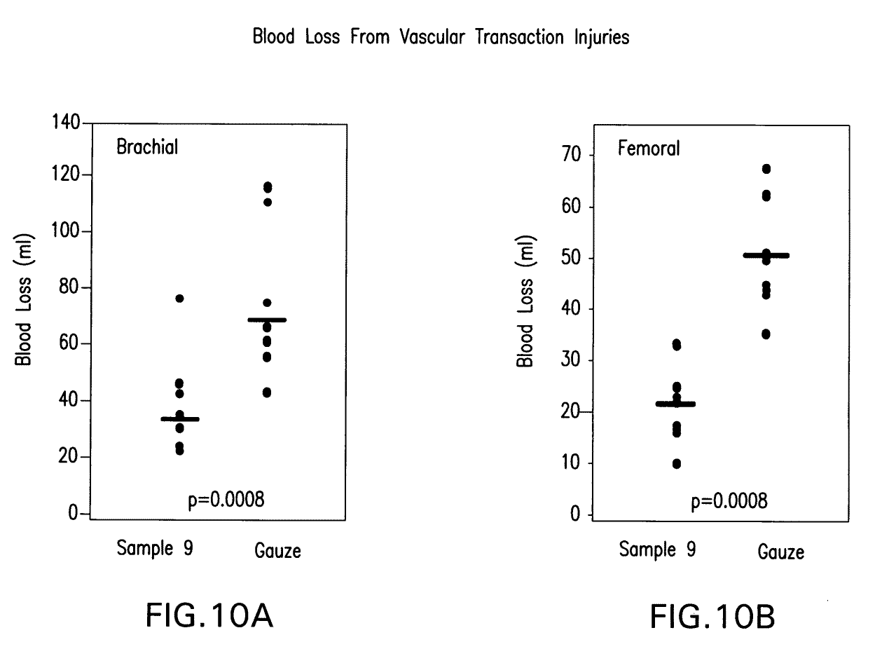

Femoral injuries were created in each animal after the completion of the brachial vasculature analysis. Bilateral femoral arteries were exposed, and then completely transected in a near simultaneous manner to initiate an exsanguinating hemorrhage. As with the brachial lacerations, the injury sites were immediately packed with either dual fiber textile material of Sample 9 or gauze. After holding pressure for six minutes the blood in the materials and shed blood was measured. An important feature of this large vessel transection model is that the animals were not in hemorrhagic shock; because the injuries were immediately packed with pressure, the mean arterial pressure was maintained in the 50 to 60 mm Hg range, end-tidal CO₂ between 35-45 mm Hg, heart between 90-100 beats/min, oxygenation between 98% and 100%. Total blood loss was not more than ˜10% of the total blood volume. Thus, blood lactate, pH and base deficient metrics were within normal physiological ranges. The total amount of blood loss with the dual fiber material was approximately half that with gauze (p=0.0006 for the comparison of brachial vasculature injuries, p=0.0005 for the femoral laceration comparisons) with both types of injuries (see FIG. 10). There was a marked tendency for gauze to pull off the haemostatic plug (to the extent there was one), while the dual fiber textile material of Sample 9 did not strongly incorporate into the haemostatic clog.

While the invention has been described in combination with embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the appended claims. All patent applications, patents, and other publications cited herein are incorporated by reference in their entireties.

What is claimed is:

1. A woven fabric having a modified crowsfoot weave pattern.

2. The woven fabric of claim 1, wherein said fabric comprises fiberglass yarn and bamboo yarn.

3. The woven fabric of claim 2, wherein said woven fabric comprises about 15.0 ounces per square yard (OSY) and has a thread ranging from about 250 to about 1000.

4. The woven fabric of claim 1, wherein the alternate yarns in the warp are 4/1 bamboo yarn.

5. The woven fabric of claim 1, wherein said thread count is the product of 38 warp yarns per inch and 20 weft yarns per inch.

6. The woven fabric of claim 1, wherein said woven fabric is capable of activating hemostatic systems in the body when applied to a wound.

7. The woven fabric of claim 1, further comprising a hemostatic agent.

8. The woven fabric of claim 7, wherein said hemostatic agent is selected from the group consisting of thrombin or a fraction containing thrombin; RL platelets, RL blood cells; fibrin, fibrinogen, and combinations thereof.

9. The woven fabric of claim 8, wherein said fraction containing thrombin further comprises one or more coagulation factors selected from the group consisting of factor XII, factor XIIa, factor XI, factor XIa, factor XIII, factor XIIIa, factor IX, factor IXa, factor VIII, factor VIIIa, factor vWF, factor V, factor Va, factor X, factor Xa, and combinations thereof.

10. The woven fabric of claim 8, wherein said fraction containing thrombin further comprises a vasoactive active agent.

11. The woven fabric of claim 10, wherein said vasoactive agent is selected from the group consisting of endothelins, thromoboxanes, NO scavengers, and combinations thereof.

12. The woven fabric of claim 1, further comprising a preservative selected from the group consisting of glycerol, propanediol, polyoxyethylene glycol (PEG), trehalose, and combinations thereof

13. The woven fabric of claim 8, wherein said thrombin or a fraction comprising thrombin comprises from about 0.01 to about 10% by weight, based on the total weight of said fabric.

14. The woven fabric of claim 13, wherein said thrombin or a fraction comprising thrombin comprises from about 0.05 to about 7% by weight, based on the total weight of said fabric.

15. The woven fabric of claim 14, wherein said thrombin or a fraction comprising thrombin comprises from about 0.1 to about 5% by weight, based on the total weight of said fabric.

16. The woven fabric of claim 8, wherein said RL platelets or said RL blood cells comprise from about 0.1 to about 20 wt %, based on the total weight of said fabric.

17. The woven fabric of claim 16, wherein said RL platelets or said RL blood cells comprise from about 1 to about 10 wt %, based on the total weight of said fabric.

18. The woven fabric of claim 17, wherein said RL platelets or said RL blood cells comprise from about 3 to about 7 wt %, based on the total weight of said fabric.

19. The woven fabric of claim 8, wherein said fibrin or fibrinogen comprises from about 0.1 to about 5 wt %, based on the total weight of said fabric.

20. The woven fabric of claim 19, wherein said fibrin or fibrinogen comprises from about 0.5 to about 2 wt %, based on the total weight of said fabric.

21. The woven fabric of claim 20, wherein said fibrin or fibrinogen comprises from about 0.75 to about 1.5 wt %, based on the total weight of said fabric.

22. The woven fabric of claim 1, further comprising one or more additional agents selected from the group consisting of skin conditioners, anti-inflammatory agents; analgesics; antimicrobial or antifungal agents; vasoconstrictors; growth factors; anti-scar agents; cauterizing agents, dehydrating agents, prothrombotic agents, and combinations thereof.

23. A woven fabric, comprising about 65 wt % fiberglass yarn and about 35 wt % bamboo yarn, said woven fabric:
(1) being about 15.0 ounces per square yard (OSY);
(2) having a thread count of about 760; and
(3) having a modified crowsfoot weave pattern.

24. The woven fabric of claim 23, wherein the alternate yarns in the warp are 4/1 bamboo yarn.

25. The woven fabric of claim 23, wherein said thread count is the product of 38 warp yarns per inch and 20 weft yarns per inch.

26. The woven fabric of claim 23, wherein said woven fabric is capable of activating hemostatic systems in the body when applied to a wound.

27. The woven fabric of claim 23, further comprising a hemostatic agent.

28. The woven fabric of claim 27, wherein said hemostatic agent is selected from the group consisting of thrombin or a fraction containing thrombin; RL platelets, RL blood cells; fibrin, fibrinogen, and combinations thereof.

29. The woven fabric of claim 28, wherein said fraction containing thrombin further comprises one or more coagulation factors selected from the group consisting of factor XII, factor XIIa, factor XI, factor XIa, factor XIII, factor XIIIa, factor IX, factor IXa, factor VIII, factor VIIIa, factor vWF, factor V, factor Va, factor X, factor Xa, and combinations thereof.

30. The woven fabric of claim 28, wherein said fraction containing thrombin further comprises a vasoactive active agent.

31. The woven fabric of claim 30, wherein said vasoactive agent is selected from the group consisting of endothelins, thromoboxanes, NO scavengers, and combinations thereof.

32. The woven fabric of claim 28, further comprising a preservative selected from the group consisting of glycerol, propanediol, polyoxyethylene glycol (PEG), trehalose, and combinations thereof.

33. The woven fabric of claim 28, wherein said thrombin or a fraction comprising thrombin comprises from about 0.01 to about 10% by weight, based on the total weight of said fabric.

34. The woven fabric of claim 33, wherein said thrombin or a fraction comprising thrombin comprises from about 0.05 to about 7% by weight, based on the total weight of said fabric.

35. The woven fabric of claim 34, wherein said thrombin or a fraction comprising thrombin comprises from about 0.1 to about 5% by weight, based on the total weight of said fabric.

36. The woven fabric of claim 28, wherein said RL platelets or said RL blood cells comprise from about 0.1 to about 20 wt %, based on the total weight of said fabric.

37. The woven fabric of claim 36, wherein said RL platelets or said RL blood cells comprise from about 1 to about 10 wt %, based on the total weight of said fabric.

38. The woven fabric of claim 37, wherein said RL platelets or said RL blood cells comprise from about 3 to about 7 wt %, based on the total weight of said fabric.

39. The woven fabric of claim 28, wherein said fibrin or fibrinogen comprises from about 0.1 to about 5 wt %, based on the total weight of said fabric.

40. The woven fabric of claim 39, wherein said fibrin or fibrinogen comprises from about 0.5 to about 2 wt %, based on the total weight of said fabric.

41. The woven fabric of claim 40, wherein said fibrin or fibrinogen comprises from about 0.75 to about 1.5 wt %, based on the total weight of said fabric.

42. The woven fabric of claim 23, further comprising one or more additional agents selected from the group consisting of skin conditioners, anti-inflammatory agents; analgesics; antimicrobial or antifungal agents; vasoconstrictors; growth factors; anti-scar agents; cauterizing agents, dehydrating agents, prothrombotic agents, and combinations thereof.

43. The woven fabric of claim 1, wherein said fabric is an article of clothing or a liner for an article of clothing.

44. The woven fabric of claim 1, wherein said fabric is part of a kit for emergency, surgical or trauma use.

45. The woven fabric of claim 23, wherein said fabric is an article of clothing or a liner for an article of clothing.

46. The woven fabric of claim 23, wherein said fabric is part of a kit for emergency, surgical or trauma use.
